(19) United States
(12) Patent Application Publication
(10) Pub. No.: US 2011/0082349 A1
(43) Pub. Date: Apr. 7, 2011

(54) MEANS AND METHODS FOR DETERMINING THE ARTERIOSCLEROTIC STENOSIS USING INFLAMMATORY BIOMARKERS

(76) Inventors: Georg Hess, Mainz (DE); Andrea Horsch, Mannheim (DE); Dietmar Zdunek, Tutzing (DE)

(21) Appl. No.: 12/966,262

(22) Filed: Dec. 13, 2010

Related U.S. Application Data
(63) Continuation of application No. PCT/EP2009/057438, filed on Jun. 16, 2009.

(30) Foreign Application Priority Data
Jun. 17, 2008 (EP) 08158415.3

(51) Int. Cl. A61B 5/00; G01N 33/68; G01N 33/92

(52) U.S. Cl. 600/300; 436/86; 436/71

(57) ABSTRACT

The present invention relates to a method for diagnosing the degree of arteriosclerotic stenosis in a subject including determining the amount of CRP or LPa in a sample of the subject and comparing the determined amount to a reference whereby the degree of arteriosclerotic stenosis is determined. The present invention also contemplates a method for identifying a subject in need of prevention or therapy of arteriosclerosis. Further, devices and kits are encompassed for carrying out the methods.

### RELATED APPLICATIONS

This application is a continuation of PCT/EP2009/057438 filed Jun. 16, 2009 and claims priority to EP 08158415.3 filed Jun. 17, 2008.

### FIELD OF THE INVENTION

The present invention is concerned with the provision of diagnostic methods relating to arteriosclerosis. Specifically, it relates to a method for diagnosing the degree of arteriosclerotic stenosis in a subject comprising determining the amount of CRP or LPa in a sample of said subject and comparing the determined amount to a reference whereby the degree of arteriosclerotic stenosis is determined. The present invention also contemplates a method for identifying a subject in need of prevention or therapy of arteriosclerosis. Further, devices and kits are encompassed for carrying out said methods.

### BACKGROUND

Arteriosclerosis is a cardiovascular disease affecting the structure of the blood vessels. It is dependent on various risk factors including smoking, hyperlipidemia, arterial hypertension, or diabetes.

Arteriosclerosis is a pathological process which in its advanced stages has severe complications caused by occlusions or stenosis of blood vessels. Prominent complications caused by the said stenosis or occlusion of blood vessels are coronary artery diseases especially angina pectoris, claudicato intennittens, myocardial infarction or stroke. These complications, however, become only clinically apparent if more than 90% of the vessel is occluded. Even in those cases they become often apparent only during exercise. Thus, under normal conditions, most of the aforementioned diseases and disorders remain undiagnosed.

The degree of stenosis due to an arteriosclerotic plaque can be classified by a scoring system which takes into account the amount of the arteriosclerotic plaques as well as the size of the remaining vessel lumen. Said scoring allows for assessing whether an individual arteriosclerotic stenosis or occlusion will have an increased likelihood of resulting in complications as referred to above.

The degree of arteriosclerotic stenosis is currently determined by cumbersome, expensive and/or invasive techniques including angiography. These techniques are inconvenient for the patient to be investigated and are time and cost-intensive from an overall health management perspective. Moreover, invasive angiography may even result in severe side-effects for the patient (C. J. Davidson, R. O. Bonow Cornorary catherization p 345; D. Pennell Cardiovascular Magnetic Resonance p 335; S. Achenbach, W. G. Daniel Computed tomography of the heart p. 255 all in Braunwald's Heart Disese 7th Ed. 2005 Elevier Publishers).

Means and methods allowing for a reliable and efficient determination of the degree of arteriosclerotic stenosis and, thus, or assessing the risk for developing severe complications of arteriosclerosis referred to above are not yet available but would be highly desirable.

The technical problem underlying the present invention could be seen as the provision of means and methods for complying with the aforementioned needs. The technical problem is solved by the embodiments characterized in the claims and herein below.

### SUMMARY OF THE INVENTION

Thus, the present invention relates to a method for diagnosing the degree of arteriosclerotic stenosis in a subject comprising:
a) determining the amount of an inflammatory marker selected from CRP or LPa in a sample of a subject; and
b) comparing the amount determined in step a) to a reference, whereby the degree of arteriosclerotic stenosis is determined.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of the subject. The method may be carried out manually or assisted by automation. Preferably, step (a) and/or (b) may in total or in part be assisted by automation, e.g., by a suitable robotic and sensory equipment for the determination in step (a) or a computer-implemented calculation in step (b).

### DETAILED DESCRIPTION OF THE INVENTION

The term “diagnosing” as used herein means assessing the arteriosclerotic load in a subject. As will be understood by those skilled in the art, such an assessment is usually not intended to be correct for all (i.e. 100%) of the subjects to be investigated. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly assessed by the method of the present invention.

The term “degree of arteriosclerotic stenosis” relates to the strength of the occlusion of vessel by an arteriosclerotic and, preferably, atherosclerotic plaque and the amount of the arteriosclerotic and, preferably, atherosclerotic plaques. The occlusion may be up to 100%, a situation were no blood flow at all is observed through the vessel. However, stenosis caused by an arteriosclerotic and, preferably, atherosclerotic plaque as used herein, preferably, results in a at least 20%, at least 50%, at least 70%, at least 80%, or at least 90% occlusion of the vessel. The degree of arteriosclerotic stenosis can also be determined by the ALS scoring system. Preferably, the degree which can be determined by the method of the present invention includes ALS classes 1 to 5 (mild stenosis), ALS classes 6 to 10 (intermediate stenosis) or ALS classes 11 to 18 (severe stenosis). Preferably, stenosis as used herein is accompanied by occlusive diseases of the peripheral arteries. More preferably, stenosis in this context is further associated with an increased risk of developing angina pectoris, claudicato intermittens or stroke wherein the risk increase is correlated with the degree of the arteriosclerotic stenosis. For scoring, the amount of peripheral arteriosclerosis is determined by angiography and classified within a score from 0 to 6. Moreover, the amount of the stenosis of both carotic arteries is measured by angiography and scored from 0 to 6. Finally, coronary sclerosis is measured and also scored from 0 to 6.

The term “subject” as used herein relates to animals, preferably mammals, and, more preferably, humans. Preferably, the subject suffers from arteriosclerosis. More preferably, the arteriosclerosis has not yet become clinically apparent.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

CRP, herein also referred to as C-reactive protein, is an acute phase protein that was discovered more than 75 years ago to be a blood protein that binds to the C-polysaccharide of pneumococci. CRP is known as a reactive inflammatory marker and is produced by a distal organ (i.e. the liver) in response or reaction to chemokines or interleukins originating from the primary lesion site. CRP has five single subunits, which are non covalently linked and assembled as a cyclic pentamer with a molecular weight of approximately 110-140 kDa. The term “CRP” preferably also relates to variants of CRP. Preferably, CRP as used herein relates to human CRP. The sequence of human CRP is well known and disclosed, preferably, in Woo 1985, J. Biol, Chem, 260 (24), 13384-13388. The level of CRP is usually low in normal individuals but can rise 100- to 200-fold or higher due to inflammation, infection or injury (Yeh 2004, Circulation 109:H-11-II-14). Preferably, the amount of CRP in a sample of a subject is determined by using CRP assays with a high sensitivity. The CRP determined by such assays is frequently also referred to as high sensitivity CRP (hsCRP). HsCRP assays are, e.g., used to predict the risk of heart disease. Suitable hsCRP assays are known in the art. A particularly preferred hsCRP assay in the context of the present invention is the Roche/Hitachi CRP (Latex) HS test with a detection limit of 0.1 mg/l. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific CRP polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc, Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific CRP polypeptides or the aforementioned types of variants as long as these fragments have the essential immunological and biological properties as referred to above. Such fragments may be, e.g., degradation products or splice variants of the CRP polypeptides. Further included are variants which differ due to posttranslational modifications such as phosphorylation or myristylation.

The term “LPa” as used herein refers to Lipoprotein (a). LPa consists of an LDL-like particle and the specific apolipoprotein(a) which is covalently bound to the apoB of the LDL like particle. LPa as used herein, preferably, refers to human LPa and variants thereof. Human LPa, preferably, has an amino acod sequence as shown in the Gene Bank database under accession umber XP 946885.2; GI: 113418363 or as disclosed by McLean 1987, Nature 330 (6144): 132-7. Assays for determining LPa are well known in the art and described in Genest 1991, American J Cardiology 67(13): 1039-1045. It is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific LPa polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific LPa polypeptides or the aforementioned types of variants as long as these fragments have the essential immunological and biological properties as referred to above. Such fragments may be, e.g., degradation products or splice variants of the LPa polypeptides. Further included are variants which differ due to posttranslational modifications such as phosphorylation or myristylation.

Determining the amount of the polypeptides referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the polypeptide. Moreover, the signal strength can, preferably, be correlated directly or indirectly (e.g. reverse-proportional) to the amount of polypeptide present in a sample. Further suitable methods comprise measuring a physical or chemical property specific for the polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

Preferably, determining the amount of a polypeptide comprises the steps of (a) contacting a cell capable of eliciting a cellular response the intensity of which is indicative of the amount of the polypeptide with the said peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g. a peptide, another polypeptide, or a small molecule. The expression or substance shall generate an intensity signal which correlates to the amount of the polypeptide.

Also preferably, determining the amount of a polypeptide comprises the step of measuring a specific intensity signal obtainable from the polypeptide in the sample. As described above, such a signal may be the signal intensity observed at an mass to charge (m/z) variable specific for the polypeptide observed in mass spectra or a NMR spectrum specific for the peptide or polypeptide.

Determining the amount of a polypeptide may, preferably, comprises the steps of (a) contacting the polypeptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, another polypeptide, nucleic acid, or small molecule, binding to the polypeptide described herein. Preferred ligands include antibodies, nucleic acids, polypeptides such as receptors or binding partners for the polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g. by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable lable prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include ³⁵S, ¹²⁵I, ³²P, ³³P and the like. A radioactive label can be detected by any method known and appropriate, e.g. a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a polypeptide may be, also preferably, determined as follows: (a) contacting a solid support comprising a ligand for the polypeptide as specified above with a sample comprising the polypeptide and (b) measuring the amount polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide, the relative amount or concentration of the said polypeptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step a) and the reference amount, it is possible to diagnose the degree of arteriosclerotic stenosis and, more specifically, to allocate the subject into the group of subjects having either mild, intermediate or severe degree of arteriosclerotic stenosis. Therefore, the reference amount is to be chosen so that either a difference or a similarity in the compared amounts allows allocation of the subjects.

Accordingly, the term “reference amounts” as used herein refers to amounts of the polypeptides which allow allocating a determined test amount into a group of subjects having either a mild, intermediate or severe degree of arteriosclerotic stenosis. Therefore, the reference may either be derived from (i) a subject known to have a mild degree of arteriosclerotic stenosis, (ii) an intermediate degree of arteriosclerotic stenosis or (iii) a severe degree of arteriosclerotic stenosis. Moreover, the reference amounts, preferably, define thresholds. Suitable reference amounts or threshold amounts may be determined by the method of the present invention from a reference sample to be analyzed together, i.e. simultaneously or subsequently, with the test sample. It is, furthermore, to be understood that the physiological amounts for CRP or LPa in a population of subjects will statistically vary. Thus, the said population will exhibit a range of amounts. Thus, statistical variations in the threshold amounts within standard deviations shall be also taken into account. Preferably, an average amount of 2 to 3 mg/L or more of CRP is indicative for a mild degree of arteriosclerotic stenosis (ALS score 1 to 5), an average amount of 6 to 7 mg/L or more is indicative for a intermediate degree of arteriosclerotic stenosis (ALS score 6 to 10) and an average amount of 12 to 13 mg/L or more is indicative for a severe degree of arteriosclerotic stenosis (ALS score 11 to 18). Likewise, an average amount of 0.08 to 0.1 mg/dL or more of LPa is indicative for a mild degree of arteriosclerotic stenosis (ALS score 1 to 5), an average amount of 0.13 to 0.15 mg/dL or more of LPa is indicative for a intermediate degree of arteriosclerotic stenosis (ALS score 6 to 10) and an average amount of 0.44 to 0.45 mg/dL or more of LPa is indicative for a severe degree of arteriosclerotic stenosis (ALS score 11 to 18). Based on the aforementioned average amounts as threshold amounts, a subject can be classified as belonging into the group of subjects having a mild, intermediate or severe degree of arteriosclerotic stenosis.

Advantageously, it has been found in accordance with the present invention that the inflammatory biomarkers CRP and LPa are independent predictors for the degree of arteriosclerotic stenosis and can be used accordingly for diagnostic purposes. This finding is surprising since the arteriosclerotic load, i.e. the indicator for the entirety of arteriosclerotic plaques in a subject, is predicted by angiogenesis markers, such as PLGF, rather than the inflammatory markers CRP or LPa. Both markers are independently or together well suited for assessing the degree arteriosclerotic stenosis for individual blood vessels in a subject and, thus, the risk for developing severe complications associated with arteriosclerosis, preferably coronary heart diseases including angina pectoris, claudicato intermittens or stroke. Accordingly, instead of using expensive and time-consuming monitoring techniques such as angiography which are associated with potentially severe side effects, the method of the present invention allows for a fast, reliable, cost-effective and safe assessment. It is to be understood that in addition to CRP or LPa, further biomarkers can be determined in the methods of the present invention.

Specifically, natriuretic peptides, preferably NT-proBNP, can be determined in order to further evaluate the degree of arteriosclerotic stenosis with respect to potential cardiovascular implications. Specifically, an average amount of NT-proBNP of at least 552 pg/ml shall be indicative for a severe degree of arteriosclerotic stenosis (ALS class 11 to 18). It is to be understood that NT-proBNP may also be determined independent of CRP or LPa for said diagnosis. Thus, the present invention also contemplates the use of NT-proBNP in a sample for diagnosing the degree of arteriosclerotic stenosis in a subject, in principle.

Moreover, it may be advantageous to determine the arteriosclerotic load, i.e. the entire amount of arteriosclerotic plaques found in a subject, prior to determine the degree of stenosis by the aforementioned methods. To this end, the amount of PlGF can be determined by a method comprising:
determining the amount of PLGF in a sample of a subject; and
calculating the ratio of the determined amount and the upper limit of normal (ULN) for PLGF, wherein
   a ratio of 1 indicates a normal arteriosclerotic load;
   a ratio less than 1 indicates a reduced arteriosclerotic load; and
   a ratio larger than 1 indicates an increased arteriosclerotic load.

The subjects are, preferably, of the same species as the subject to be investigated and, even more preferably, of the same ethnical background. How to determine the ULN is well known in the art and has been carried out for various polypeptides already. Particularly, a suitable range for an ULN can be derived from the amounts found between the 25^{th} and 75^{th} percentiles. More preferably, the said ULN for PLGF is between 7 and 10 pg/ml, most preferably, 8 pg/ml. It will be understood that the ULN may vary due to statistics. Thus, variations in the ULN amount within standard deviations shall be also taken into account.

Calculating as used herein refers to assessing the ratio of the amount of PLGF determined in the sample of the subject and the ULN. If the amount of PLGF determined in the sample is larger than the ULN, the ratio will be larger than one (1). The ratio will be less than 1, if the determined amount for PLGF is less than the ULN. The ratio will be 1 for an amount of PLGF determined in the sample being identical to the ULN. Moreover, it is to be understood that a ratio of I indicates a normal arteriosclerotic load and, thus, normal risk for developing the severe complications associated with arteriosclerosis. A ratio less than 1 indicates a reduced arteriosclerotic load and, consequently, a reduced risk for developing the severe complications associated with arteriosclerosis. A ratio larger than 1 indicates an increased arteriosclerotic load and, as a result thereof, an increased risk for developing the severe complications associated with arteriosclerosis.

The present invention, thus, in a preferred embodiment, provides for a method comprising the determination of the arteriosclerotic load based on PlGF as specified above and, as a subsequent step for those subjects having a high arteriosclerotic load, the determination of the degree of arteriosclerotic stenosis. Also preferably, cardiovascular implications may be further judged by determining a cardiac marker, preferably, NT-proBNP or Troponin T or I as set forth above.

The definitions and explanations of the terms given above apply mutatis mutandis for the preferred methods, the devices and kits referred to in the following.

The present invention further relates to a method for identifying a subject in need of therapy of arteriosclerosis, the method comprises the steps of the aforementioned method and the further step of identifying a subject in need of therapy of arteriosclerosis based on the degree of arteriosclerotic stenosis.

The term “therapy of arteriosclerosis” refers to drug-based therapies and to interventions such as angioplasty procedures, stent implantation, laser based revascularization, ballon dilatation, or arterial surgery. Preferred therapies are drugs against hypertension, lipid lowering drugs, preferably statins, aspirin, clopidogrel, beta-blockers, ACE inhibitors, anticoagulation drugs, angiogenic drugs for revascularization, estrogen replacement, drugs against diabetes. Therapy also includes life style recommendations, preferably, avoiding smoking and exercise.

Preferably, wherein said therapy for mild arteriosclerotic stenosis is providing life style recommendations, for intermediate arteriosclerotic stenosis is a drug based therapy selected from administration of drugs against hypertension, lipid lowering drugs, preferably statins, aspirin, clopidogrel, beta-blockers, ACE inhibitors, anticoagulation drugs, angiogenic drugs for revascularization, estrogen replacement, drugs against diabetes, and for severe arteriosclerotic stenosis is an intervention based therapy selected from angioplasty procedures, stent implantation, laser based revascularization, ballon dilatation, or arterial surgery.

It will be understood that the therapy for mild arteriosclerotic stenosis may be also applied in addition for the intermediate and severe cases and that the therapy for intermediate cases may also be applied for the severe cases.

The present invention also relates to a device for diagnosing the degree of arteriosclerotic stenosis of a subject comprising:
a) means for determining CRP or LPa in a sample of said subject; and
b) means for comparing the amount determined by the means of a) with a reference, whereby the degree of arteriosclerotic stenosis is to be diagnosed.

The term “device” as used herein relates to a system of means comprising at least the aforementioned means operatively linked to each other as to allow the prediction. Preferred means for determining the amount of CRP or LPa polypeptide as well as means for carrying out the comparison are disclosed above in connection with the method of the invention. How to link the means in an operating manner will depend on the type of means included into the device. For example, where means for automatically determining the amount of the polypeptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the polypeptides in an applied sample and a computer unit for processing the resulting data for the evaluation. The computer unit, preferably, comprises a database including the stored threshold reference amounts or values derived therefrom recited elsewhere in this specification as well as a computer-implemented algorithm for carrying out a comparison of the determined amounts for the polypeptides and the stored reference amounts of the database, Computer-implemented as used herein refers to a computer-readable program code tangibly included into the computer unit. The person skilled in the art will realize how to link the means without further ado. Preferred devices are those which can be applied without the particular knowledge of a specialized clinician, e.g., electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the polypeptides. Plasmon surface resonace devices, NMR spectrometers, mass-spectrometers etc.) and/or evaluation units/devices referred to above in accordance with the method of the invention.

Finally, the present invention relates to a kit adapted for carrying out the aforementioned methods comprising:
a) means for determining CRP or LPa in a sample of said subject; and
b) means for comparing the determined amount of the means of a) with a reference, whereby the degree of arteriosclerotic stenosis is to be diagnosed.

The term “kit” as used herein refers to a collection of the aforementioned means, preferably, provided separately or within a single container. The components of the kit may be comprised by separate vials (i.e. as a kit of separate parts) or provided in a single vial. Moreover, it is to be understood that the kit of the present invention is to be used for practising the methods referred to herein above. It is, preferably, envisaged that all components are provided in a ready-to-use manner for practising the methods referred to above. Further, the kit preferably contains instructions for carrying out the said methods. The instructions can be provided by a user's manual in paper- or electronic form. For example, the manual may comprise instructions for interpreting the results obtained when carrying out the aforementioned methods using the kit of the present invention.

All references cited in this specification are herewith incorporated by reference with respect to their entire disclosure content and the disclosure content specifically mentioned in this specification.

The following examples shall merely illustrate the invention. They shall not be construed, whatsoever, to limit the scope of the invention.

### Example 1

### Determination of Angiogenesis and Inflammatory Biomarkers in Patients Suffering from Peripheral Arterial Occlusive Disease

50 patients suffering from a peripheral arterial occlusive disease were analyzed for the plasma levels for the angiogenesis biomarkers PLGF, soluble (s)Flt-1, and endoglin as well as the inflammatory markers CRP and LPa.

Plasma levels of PlGF, sFLT1, and Endoglin were determined using the commercially available Immunoassays “Quantikine” (Catalog numbers DVR100B, DPG00 and DNDG00) from R & D Systems, USA. CRP and LPa were determined by the CRP (Latex) HS assay from Roche Diagnostics, Germany, and for LPa as described in Genest 1991, American J Cardiology 67(13): 1039-45.

The inflammatory biomarkers CRP and LPa, both, were found to correlate with the degree of arteriosclerotic stenosis while such a correlation was absent for the angiogenesis markers. The degree of arteriosclerosis was also determined by the ASL scoring system.

The results are summarized in the Tables 1 and 2, below.

### Example 2

### CRP Levels Decrease in Patients after Reperfusion Due to Stent Implantation

Plasma levels of CRP were determined for 18 patients suffering from arteriosclerotic coronary vessel stenosis prior and 30 days after a stent implantation surgery was carried out. The plasma levels were determined as described in Example 1.

After reperfusion and stent implantation, the CRP levels decreased.

The results are shown in Table 3, below.

TABLE 1
Angiogenesis marker show no significant differences

| | PlGF/pg/ml | | | sFlt-1/pg/ml | | |
|---|---|---|---|---|---|---|
| | ASL | ASL | ASL | ASL | ASL | ASL |
| | Score | Score | Score | Score | Score | Score |
| | 1-5 | 6-10 | 11-18 | 1-5 | 6-10 | 11-18 |
| Median | 17.20 | 18.80 | 18.60 | 95.30 | 94.60 | 98.00 |

| | Endoglin/ng/ml | | |
|---|---|---|---|
| | ASL | ASL | ASL |
| | Score | Score | Score |
| | 1-5 | 6-10 | 11-18 |
| Median | 4.10 | 4.19 | 3.72 |

TABLE 2
CRP and LPa allow for allocation into the ASL classes

| | hsCRP/mg/L | | | LPa/mg/dL | | |
|---|---|---|---|---|---|---|
| | ASL | ASL | ASL | ASL | ASL | ASL |
| | Score | Score | Score | Score | Score | Score |
| | 1-5 | 6-10 | 11-18 | 1-5 | 6-10 | 11-18 |
| Median | 2.72 | 6.66 | 12.88 | 0.09 | 0.14 | 0.45 |

TABLE 3
CRP levels decrease after stent implantation

| | hsCRP/mg/L at day 0 | hsCRP/mg/L at day 30 |
|---|---|---|
| Median | 3.58 | 2.55 |

What is claimed is:

1. A method for diagnosing a degree of arteriosclerotic stenosis in a subject comprising
determining an amount of lipoprotein(a) (LPa) in a sample from the subject and
comparing the amount determined with a reference amount of LPa, wherein the reference amount is derived from a subject known to have a mild degree of arteriosclerotic stenosis, an intermediate degree of arteriosclerotic stenosis or a severe degree of arteriosclerotic stenosis.

2. A method for diagnosing a degree of arteriosclerotic stenosis in a subject comprising
determining an amount of lipoprotein(a) (LPa) in a sample from the subject and
comparing the amount determined with a reference amount of LPa, wherein a determined LPa in an amount of 0.08 to 0.1 mg/dL or more is indicative for a mild degree of arteriosclerotic stenosis (ALS score 1 to 5), a determined LPa in an amount of 0.13 to 0.15 mg/dL or more is indicative for a intermediate degree of arteriosclerotic stenosis (ALS score 6 to 10), and a determined LPa in an amount of 0.44 to 0.45 mg/dL or more is indicative for a severe degree of arteriosclerotic stenosis (ALS score 11 to 18).

3. A method for diagnosing a degree of arteriosclerotic stenosis in a subject comprising
determining an amount of C-reactive protein (CRP) in a sample from the subject and
comparing the amount determined with a reference amount of CRP, wherein a determined CRP in an amount of 2 to 3 mg/L or more is indicative for a mild degree of arteriosclerotic stenosis (ALS score 1 to 5), a determined CRP in an amount of 6 to 7 mg/To or more is indicative for a intermediate degree of arteriosclerotic stenosis (ALS score 6 to 10), and a determined CRP in an amount of 12 to 13 mg/L or more is indicative for a severe degree of arteriosclerotic stenosis (ALS score 11 to 18).

4. A method for identifying a subject in need of therapy of arteriosclerosis, the method comprising the steps of the method of claim 1 and the further step of identifying a subject in need of therapy of arteriosclerosis based on the degree of arteriosclerotic stenosis.

5. The method of claim 4, wherein said therapy for mild arteriosclerotic stenosis is providing life style recommendations, for intermediate arteriosclerotic stenosis is a drug based therapy selected from administration of drugs against hypertension, lipid lowering drugs, preferably statins, aspirin, clopidogrel, beta-blockers, ACE inhibitors, anticoagulation drugs, angiogenic drugs for revascularization, estrogen replacement, drugs against diabetes, and for severe arteriosclerotic stenosis is an intervention based therapy selected from angioplasty procedures, stent implantation, laser based revascularization, balloon dilatation, or arterial surgery.

6. A method for identifying a subject in need of therapy of arteriosclerosis, the method comprising the steps of the method of claim 3 and the further step of identifying a subject in need of therapy of arteriosclerosis based on the degree of arteriosclerotic stenosis.

7. The method of claim 4, wherein said therapy for mild arteriosclerotic stenosis is providing life style recommendations, for intermediate arteriosclerotic stenosis is a drug based therapy selected from administration of drugs against hypertension, lipid lowering drugs, preferably statins, aspirin, clopidogrel, beta-blockers, ACE inhibitors, anticoagulation drugs, angiogenic drugs for revascularization, estrogen replacement, drugs against diabetes, and for severe arteriosclerotic stenosis is an intervention based therapy selected from angioplasty procedures, stent implantation, laser based revascularization, balloon dilatation, or arterial surgery.

8. A device for diagnosing the degree of arteriosclerotic stenosis in a subject according to the method of claim 1 comprising:
means for determining lipoprotein(a) (LPa) in a sample from the subject; and
means for comparing the amount determined to a reference amount of LPa.

9. A device for diagnosing the degree of arteriosclerotic stenosis in a subject according to the method of claim 3 comprising:
means for determining C-reactive protein (CRP) in a sample from the subject and
means for comparing the amount determined to a reference amount of CRP.

10. A kit adapted for diagnosing the degree of arteriosclerotic stenosis in a subject according to the method of claim 1 comprising
instructions for carrying out the method,
means for determining lipoprotein(a) (LPa) in a sample from the subject and
means for comparing the amount determined to a reference amount of LPa.

11. A kit adapted for diagnosing the degree of arteriosclerotic stenosis in a subject according to the method of claim 1 comprising
instructions for carrying out the method,
means for determining C-reactive protein (CRP) in a sample from the subject and
means for comparing the amount determined to a reference amount of CRP.
